(19) United States
(12) Patent Application Publication
(10) Pub. No.: US 2007/0233262 A1
(43) Pub. Date: Oct. 4, 2007

(54) ARTICULATING SPINAL PROSTHESIS

(76) Inventors: Uri Arnin, Kiryat Tivon (IL); Shai Fleischer, Haifa (IL)

Correspondence Address: David Klein;DEKEL PATENT LTD, Beit HaRof'im, 18 Menuha VeNahala Street, Room 27, REHOVOT, omitted

(21) Appl. No.: 11/692,194

(22) Filed: Mar. 28, 2007

Related U.S. Application Data
(60) Provisional application No. 60/788,188, filed on Mar. 31, 2006.

(51) Int. Cl. A61F 2/44

(52) U.S. Cl. 623/17.15; 623/17.16

(57) ABSTRACT

An endoprosthesis including a first articulating member formed with a generally concave articulating surface, a second articulating member formed with a generally convex articulating surface which articulates with the concave articulating surface, the first and second articulating members each having a generally planar attachment surface for attachment to spine structure, and limiting members formed in each of the convex and concave articulating surfaces that mate with each other and limit the amount of relative movement between the convex and concave articulating surfaces.

### CROSS-REFERENCE TO RELATED APPLICATIONS

This application claims priority under 35 USC §119 to U.S. Provisional Patent Application Ser. No. 60/788,188, filed on Apr. 4, 2006, which is incorporated herein by reference.

### FIELD OF THE INVENTION

The present invention relates to spinal prostheses and particularly prostheses for disc or partial spinal disc replacement.

### BACKGROUND OF THE INVENTION

Spinal disc or partial spinal disc replacement is a common procedure in the US and Europe. By replacing a disc and not merely fusing vertebrae, which is the common alternative practice to disc replacement, the mobility of the patient's adjacent discs may be preserved and thus may delay the onset of arthritic changes to adjacent vertebrae.

PCT published patent application WO 2005/084590, the disclosure of which is incorporated herein by reference, describes a spinal endoprosthesis for disc or partial spinal disc replacement. The endoprosthesis including a first articulating member formed with a generally concave articulating surface, and a second articulating member formed with a generally convex articulating surface which articulates with the concave articulating surface, the first and second articulating members each having a generally planar attachment surface for attachment to spine structure, wherein the first articulating member includes a contour defined by a cutting surface that cuts through a portion of the first articulating member at a non-zero angle relative to its attachment surface, and the second articulating member includes a contour defined by a cutting surface that cuts through a portion of the second articulating member at a non-zero angle relative to its attachment surface. The attachment surfaces may include protrusions to improve attachment to the spine structure. The first articulation member may include a plurality of bulbous articulation surfaces which articulate with the second articulation member. An elastomeric member may be positioned between the first and second articulation members. One of the articulation members may include a varying height element whose protrusion from its attachment surface is adjustable.

### SUMMARY OF THE INVENTION

The present invention is directed to a spinal prosthesis that provides anti-slip features to the spinal endoprosthesis of WO 2005/084590, as is described more in detail hereinbelow.

There is thus provided in accordance with an embodiment of the present invention an endoprosthesis including a first articulating member formed with a generally concave articulating surface, a second articulating member formed with a generally convex articulating surface which articulates with the concave articulating surface, the first and second articulating members each having a generally planar attachment surface for attachment to spine structure, and limiting members formed in each of the convex and concave articulating surfaces that mate with each other and limit the amount of relative movement between the convex and concave articulating surfaces.

In accordance with a non-limiting embodiment of the present invention the limiting members include at least one ridge protruding from one of the articulating surfaces that sits and moves in at least one groove formed in the other the articulating surface. The limiting members may include a pair of generally orthogonal ridges protruding from one of the articulating surfaces that sit and move in corresponding generally orthogonal grooves formed in the other the articulating surface. The ridge(s) and groove(s) may be trapezoidal in cross section

In accordance with another non-limiting embodiment of the present invention the limiting members include at least one cord that flexibly connects the articulating surfaces to each other.

In accordance with another non-limiting the convex articulating surface includes a plurality of bulbous articulation surfaces, and the limiting members are formed in at least one of the bulbous articulation surfaces.

The endoprosthesis may further include an elastomeric member positioned between the first and second articulation members. Alternatively, one of the articulation members may include a varying height element whose protrusion from its attachment surface is adjustable.

### BRIEF DESCRIPTION OF THE DRAWINGS

In the drawings:

FIGS. 1A and 1B are simplified perspective top view and exploded view illustrations, respectively, of an endoprosthesis, constructed and operative in accordance with an embodiment of the present invention;

FIGS. 2A-2C are simplified illustrations of an endoprosthesis, constructed and operative in accordance with another embodiment of the present invention, wherein two articulation members articulate against a third articulation member;

FIGS. 3A-3C are simplified illustrations of an endoprosthesis, constructed and operative in accordance with yet another embodiment of the present invention, which may be similar in construction to the endoprosthesis of FIGS. 2A-2C, except that the third articulation member may be constructed as a hollow member;

FIGS. 4A-4C are simplified illustrations of an endoprosthesis, constructed and operative in accordance with still another embodiment of the present invention, wherein a first articulation member comprises a plurality of bulbous articulation surfaces that articulate with a second articulation member;

FIGS. 5A-5C are simplified illustrations of an embodiment similar to that of FIGS. 4A-4C, but with an additional elastomeric member between the first and second articulation members; and

FIGS. 6A-6C are simplified illustrations of an endoprosthesis, constructed and operative in accordance with another embodiment of the present invention, with male and female members associated with the articulation members.

### DESCRIPTION OF EMBODIMENTS

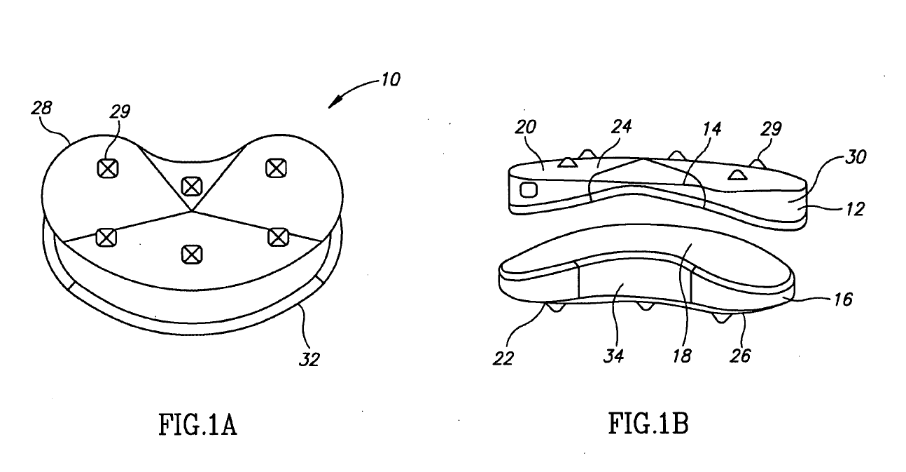

Reference is now made to FIGS. 1A and 1B, which illustrate a vertebral disc endoprosthesis 10, constructed and operative in accordance with an embodiment of the present invention. Endoprosthesis 10 may be fashioned from any combination of materials used for spinal prostheses, such as but not limited to, ceramic, aluminum oxide, zirconium oxide, metal, metal alloy, cobalt-chromium-tungsten-nickel alloy, cobalt-chromium-manganese alloy, cobalt-chromium alloy, cobalt-chromium-molybdenum alloy, chromium-nickel-manganese alloy, powder metal alloy, 316L stainless steel, Ti 6A1-4V ELI (nominally 6 percent aluminum, 4 percent vanadium, balance by weight titanium, extra-low interstitial grade), polymer, polyurethane, polyethylene, wear resistant polyethylene, cross-linked polyethylene, thermoplastic elastomer, biomaterial, polycaprolactone, diffusion hardened material, Ti-13-13, zirconium alloy, niobium alloy, with or without a porous coating system, hydrophilic coating system, hydroxyapatite coating system, and tri-calcium phosphate coating system.

In accordance with a non-limiting embodiment of the invention, endoprosthesis 10 may include a first articulating member 12 formed with a generally concave articulating surface 14, and a second articulating member 16 formed with a generally convex articulating surface 18 which articulates with concave articulating surface 14. The first and second articulating members 12 and 16 may have generally planar attachment surfaces 20 and 22, respectively, for attachment to structure of the spine. The attachment surface 20 may be generally flat, being defined by a plane 24 (e.g., a horizontal plane) that cuts through and truncates a portion of first articulating member 12 distanced from articulating surface 14. Likewise, the attachment surface 22 may be generally flat, being defined by another plane 26 (e.g., a horizontal plane) that cuts through and truncates a portion of second articulating member 16 distanced from articulating surface 18.

The attachment surfaces 20 and 22 may include spikes or other protrusions 29 to improve the attachment to the spine structure.

In accordance with a non-limiting embodiment of the invention, the first articulating member 12 includes a contour 28 defined by a cutting surface 30 that cuts through another portion of first articulating member 12 at a non-zero angle relative to the plane 24. Similarly, the second articulating member 16 includes a contour 32 defined by a cutting surface 34 that cuts through another portion of second articulating member 16 at a non-zero angle relative to the plane 26. For example, the cutting surfaces 30 and 34 may cut a “half-moon” or “banana” shape that defines contours 28 and 32 of first and second articulating members 12 and 16, respectively.

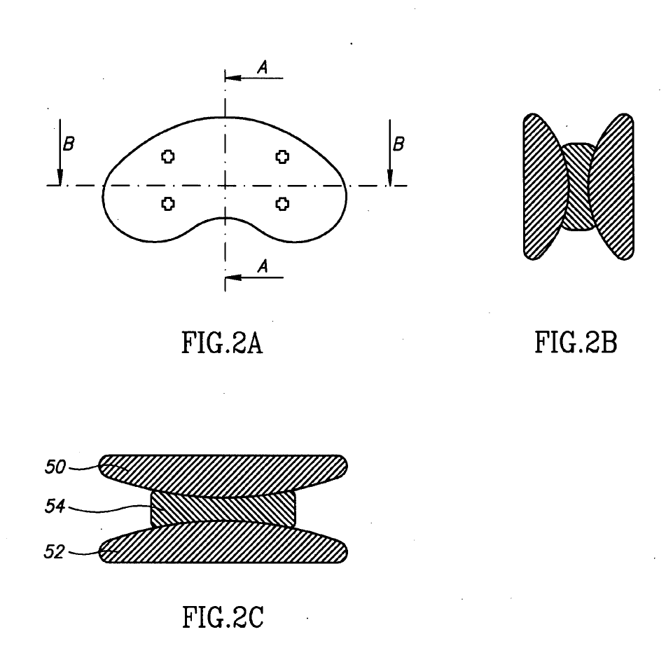

Reference is now made to FIGS. 2A-2C, which illustrate an endoprosthesis, constructed and operative in accordance with another embodiment of the present invention. In this embodiment, two articulation members 50 and 52 with generally planar attachment surfaces on their outer surfaces articulate against a third articulation member 54 on their inner surfaces. In the illustrated embodiment, the inner surfaces of articulation members 50 and 52 are shown as having a convex shape and opposing articulating surfaces of third articulation member 54 have a concave shape. It is to be understood, however, that any combination of opposing concave and convex surfaces according to this embodiment would be in the scope of the present invention. For example, the inner surfaces of articulation members 50 and 52 may have a concave shape and the opposing articulating surfaces of third articulation member 54 may have a convex shape.

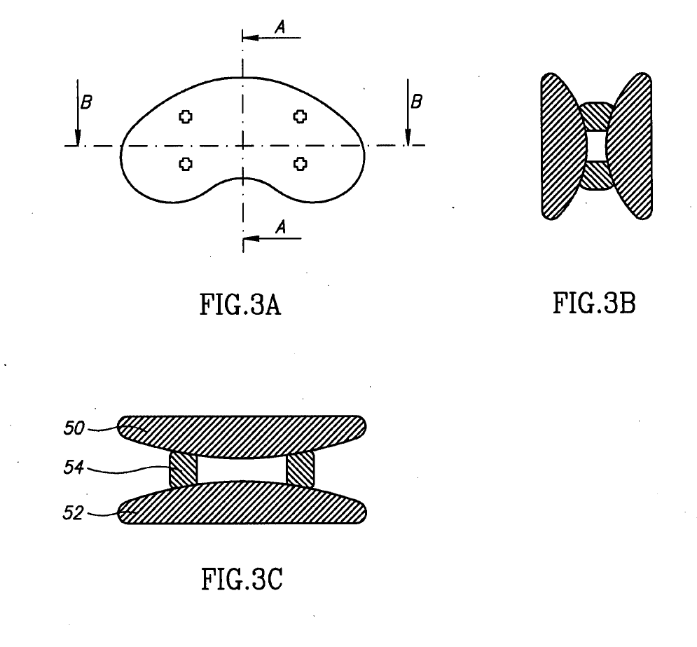

Reference is now made to FIGS. 3A-3C, which illustrate an endoprosthesis, constructed and operative in accordance with yet another embodiment of the present invention. The endoprosthesis of FIGS. 3A-3C may be similar in construction to the endoprosthesis of FIGS. 2A-2C, except that the third articulation member 54 may be constructed as a hollow member.

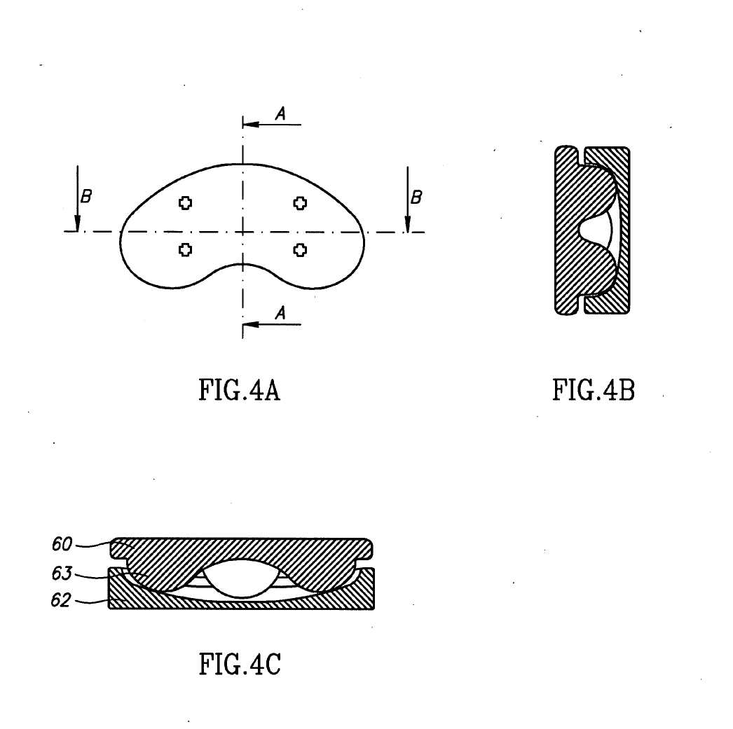

Reference is now made to FIGS. 4A-4C, which illustrate an endoprosthesis, constructed and operative in accordance with still another embodiment of the present invention. In this embodiment, a first articulation member 60 articulates with a second articulation member 62. The first articulation member 60 may comprise a plurality of bulbous articulation surfaces 63 which articulate with the generally concave second articulation member 62. The plurality of bulbous articulation surfaces 63 may provide additional degrees of rotational or other movement, such as but not limited to, flexion, extension and lateral bending.

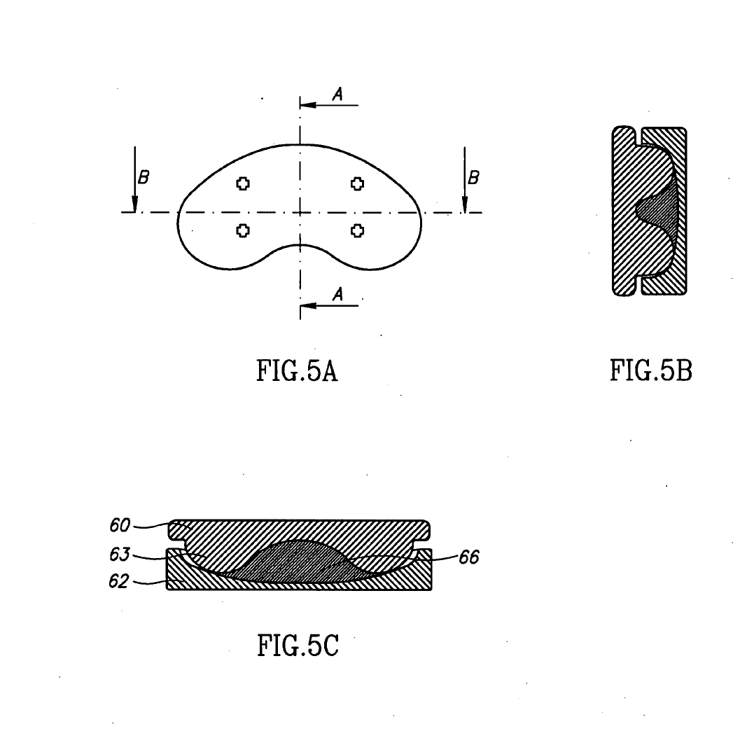

Reference is now made to FIGS. 5A-5C, which illustrate an embodiment similar to that of FIGS. 4A-4C, but with an additional elastomeric member 66 between first and second articulation members 60 and 62. Elastomeric member 66 is configured to add a cushioning effect to endoprosthesis 10.

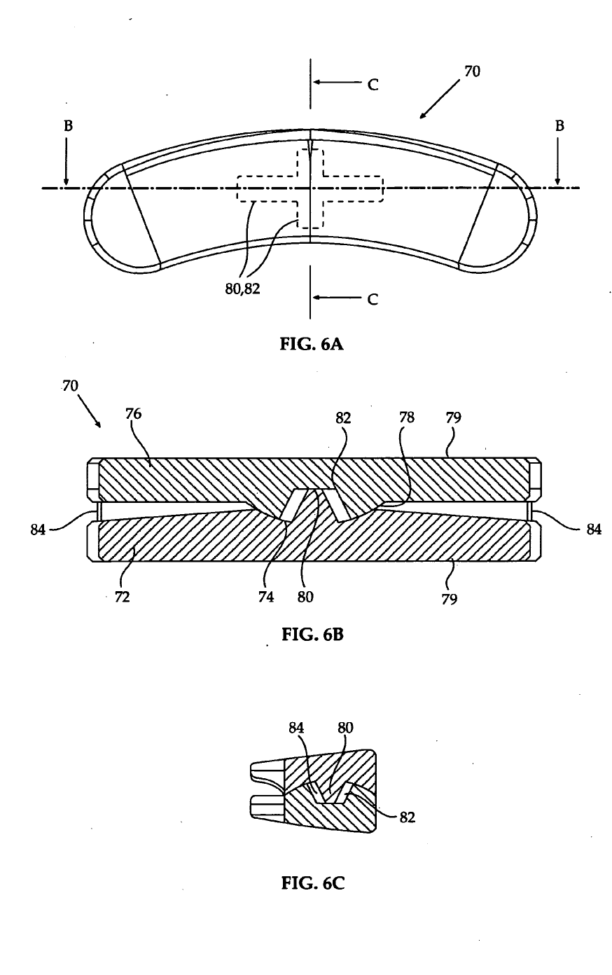

Reference is now made to FIGS. 6A-6C, which illustrate an endoprosthesis 70 with movement or anti-slipping limiting structure, constructed and operative in accordance with an embodiment of the present invention.

Endoprosthesis 70 may include a first articulating member 72 formed with a generally concave articulating surface 74, a second articulating member 76 formed with a generally convex articulating surface 78 which articulates with the concave articulating surface 74. First and second articulating members 72 and 76 each have a generally planar attachment surface 79 for attachment to spine structure (not shown).

Limiting members are formed in the convex and concave articulating surfaces 78 and 74, respectively, which mate with each other and limit the amount of relative movement between the convex and concave articulating surfaces 78 and 74.

In accordance with a non-limiting embodiment of the present invention the limiting members include one or more ridges 80 protruding from one of the articulating surfaces that sit and move in one or more grooves 82 formed in the other articulating surface. The limiting members may include a pair of generally orthogonal ridges 80 protruding from one of the articulating surfaces that sit and move in corresponding generally orthogonal grooves 82 formed in the other the articulating surface. The ridges 80 and grooves 82 may be trapezoidal in cross section.

Alternatively or additionally, the limiting members include one or more cords 84 (strings, wires, cables and the like) that flexibly connects the articulating surfaces to each other (e.g., at the sides of the endoprosthesis, in the grooves 82 or any other suitable place).

The limiting members of the invention may be implemented in any of the endoprosthesis embodiments of the present invention. For example, the limiting members may be formed in one or more of the bulbous articulation surfaces of the embodiment of FIGS. 4A-4C.

The limiting members help prevent one articulation member slipping away from a second articulation member. Slipping or disengagement of the articulation members from each other may occur, for example, during initial insertion of the implant or as a result of shear forces during installment or use. The limiting members may aid in finding the correct place of one articulation member once the other articulation member is in place. The limiting members may limit and control the amount of longitudinal and/or lateral bending and/or flexion.

Although the invention has been described in conjunction with specific embodiments thereof, it is evident that many alternatives, modifications and variations will be apparent to those skilled in the art. Accordingly, it is intended to embrace all such alternatives, modifications and variations.

What is claimed is:

1. An endoprosthesis comprising:
a first articulating member formed with a generally concave articulating surface;
a second articulating member formed with a generally convex articulating surface which articulates with said concave articulating surface, said first and second articulating members each having a generally planar attachment surface for attachment to spine structure; and
limiting members formed in each of said convex and concave articulating surfaces that mate with each other and limit the amount of relative movement between said convex and concave articulating surfaces.

2. The endoprosthesis according to claim 1, wherein said limiting members comprise at least one ridge protruding from one of the articulating surfaces that sits and moves in at least one groove formed in the other the articulating surface.

3. The endoprosthesis according to claim 1, wherein said limiting members comprise a pair of generally orthogonal ridges protruding from one of the articulating surfaces that sit and move in corresponding generally orthogonal grooves formed in the other the articulating surface.

4. The endoprosthesis according to claim 1, wherein said limiting members comprise at least one cord that flexibly connects the articulating surfaces to each other.

5. The endoprosthesis according to claim 1, wherein said convex articulating surface comprises a plurality of bulbous articulation surfaces, and wherein said limiting members are formed in at least one of said bulbous articulation surfaces.

6. The endoprosthesis according to claim 1, further comprising an elastomeric member positioned between said first and second articulation members.

7. The endoprosthesis according to claim 1, wherein one of said articulation members comprises a varying height element whose protrusion from its attachment surface is adjustable.

8. The endoprosthesis according to claim 2, wherein said at least one ridge and said at least one groove are trapezoidal in cross section.
